(19) United States
(12) Patent Application Publication
(10) Pub. No.: US 2026/0354374 A1
(43) Pub. Date: Nov. 12, 2026

(54) IMAGE-GUIDED TEMPERATURE FEEDBACK CONTROL FOR DAMAGE FREE MAGNETIC REWARMING AND RECOVERY OF CRYOPRESERVED TISSUE

(71) Applicant: THE JOHNS HOPKINS UNIVERSITY, Baltimore, MD (US)

(72) Inventors: Anirudh SHARMA, Baltimore, MD (US); Robert IVKOV, Ellicott City, MD (US)

(21) Appl. No.: 19/122,891

(22) PCT Filed: Oct. 23, 2023
(86) PCT No.: PCT/US2023/035675

§ 371 Date: Apr. 21, 2025

Related U.S. Application Data
(60) Provisional application No. 63/383,559, filed on Nov. 14, 2022.

(51) Int. Cl. A01N 1/168; A01N 1/125; A01N 1/143; A01N 1/162; G01N 24/08

(52) U.S. Cl. CPC A01N 1/168; A01N 1/125; A01N 1/143; A01N 1/162; G01N 24/08

(57) ABSTRACT

A system and method for preserving and recovering biological tissue are disclosed. The system and method includes perfusing an ex vivo organ with a biological tissue preservation composition including a magnet-responsive colloid, scanning the ex vivo organ using magnetic particle imaging, and cooling the perfused ex vivo organ to a first temperature. The method includes scanning an ex vivo organ using magnetic particle imaging to generate images representing a distribution of magnet-responsive colloid within the ex vivo organ. The method also includes co-registering the magnetic particle imaging with MRI and/or CT imaging of the biological tissue, generating 3D computational models for the biological tissue from co-registered images, applying an effective alternating magnetic field informed by temperature feedback control to the ex vivo organ using a magnetic field coil external to the ex vivo organ, and warming the ex vivo organ from the first temperature to a second temperature.

### CROSS-REFERENCE TO RELATED APPLICATIONS

This application is the national stage entry of International Patent Application No. PCT/US2023/035675, filed on Oct. 23, 2023, and published as WO 2024/107310 A1 on May 23, 2024, which claims the benefit of U.S. Provisional Patent Application No. 63/383,559, filed on Nov. 14, 2022, which are hereby incorporated by reference in their entireties.

### TECHNICAL FIELD

The present teachings relate generally to medical imaging and, more particularly, to image guided temperature control related to preservation to preserved tissue.

### BACKGROUND

End stage liver disease, including cancer, is a significant cause of mortality worldwide. Liver transplantation remains the best treatment option to extend life expectancy and quality of life, and is often the sole treatment option for many patients. Unfortunately, there remains an enormous and growing gap between organ supply and demand. Developing technologies and methods to extend organ storage times can quickly alleviate this gap and enable transportation of organs for transplantation over greater distances. Additionally, as the liver has unique regenerative properties, these technologies could be extended to hepatic sub-systems, such as hepatocytes, precision cut liver slices, liver lobes, and extended criteria donors, for example, fatty livers, potentially having a tremendous impact on tissue regeneration, liver pathophysiology and pharmacotoxicology research.

Cryopreservation of cells and tissues has shown tremendous potential and success towards long-term storage. Organ and tissue storage time increases at cryogenic temperatures because cellular metabolic activity and molecular diffusion are dramatically reduced. Cryopreservation technology can be extended to whole vascularized organs through a process called vitrification, such as rapid cooling of organs, perfused with cryoprotectants, to cryogenic temperatures (−150 °C), where they can be stored in a glassy state without damage from ice. However, before these organs can be recovered for transplantation they need to be rewarmed rapidly and uniformly from cryogenic temperatures, which remains a challenge, especially at the scale of whole organs. This is because warming rates required to prevent damage from ice-crystallization are typically an order of magnitude higher (~>100 °C/min) than cooling rates of cryoprotectants used for organ preservation. Additionally, the organ needs to be rewarmed uniformly to minimize thermal gradients to avoid cracking arising from thermomechanical stresses.

Thus, a method to rewarm organs rapidly and uniformly is needed to recover organs damage-free from the cryopreserved state.

### SUMMARY

The following presents a simplified summary in order to provide a basic understanding of some aspects of one or more embodiments of the present teachings. This summary is not an extensive overview, nor is it intended to identify key or critical elements of the present teachings, nor to delineate the scope of the disclosure. Rather, its primary purpose is merely to present one or more concepts in simplified form as a prelude to the detailed description presented later.

One general aspect of the present disclosure includes a method for preserving and recovering biological tissue. The method includes perfusing an ex vivo organ with a biological tissue preservation composition or mixture which may include a magnet-responsive colloid, scanning the ex vivo organ using magnetic particle imaging, cooling the perfused ex vivo organ to a first temperature. The method also includes scanning an ex vivo organ using magnetic particle imaging to generate one or more images representing a distribution of magnet-responsive colloid within the ex vivo organ. The method also includes co-registering the magnetic particle imaging with MRI and/or CT imaging of the biological tissue. The method also includes generating 3D computational models for the biological tissue from co-registered images. The method also includes applying an effective alternating magnetic field to the ex vivo organ using a magnetic field coil that is external to the ex vivo organ. The method also includes warming the ex vivo organ from the first temperature to a second temperature.

Implementations of the method for preserving and recovering biological tissue may include where the biological tissue preservation composition may include organic solvents including ethylene glycol, sugars, antifreeze proteins, a magnet-responsive colloid, or a combination thereof. The ethylene glycol is present in the biological tissue preservation composition in an amount of from about 10% to about 50% based on a total weight of the biological tissue preservation composition. The magnet-responsive colloid may include a plurality of magnetic particles having a particle size of from about 1 nm to about 10 microns. The method for preserving and recovering biological tissue may include storing the ex vivo organ at or below the first temperature. The method for preserving and recovering biological tissue may include, maintaining the ex vivo organ at or below the first temperature, and transporting the ex vivo organ from a first geographical location to a second geographical location. The perfused ex vivo organ is cooled to the first temperature at a rate of from about 0.5 °C/min to about 20 °C/min. The perfused ex vivo organ is warmed to the second temperature at a rate greater than 50 °C/min. The first temperature is from about 0 °C to about −150 °C. The second temperature is from about 0 °C to about 40 °C. The method for preserving and recovering biological tissue may include, performing one or more finite element modeling-based simulations of coupled electromagnetic and heat transfer in 3D models of the biological tissue using the one or more generated images, and inputting optimization results of the one or more simulations including proportional, integral and derivative gains into a multi-input multi-output (MIMO) adaptive temperature feedback control system to control one or more parameters of the magnetic field. The one or more parameters of the magnetic field may include frequency, amplitude, power, spatial and temporal localization of the effective alternating magnetic field region relative to one or more biological tissue boundaries or a combination thereof. The method for preserving and recovering biological tissue may include monitoring temperature of the ex vivo organ while applying the magnetic field, and modulating an amplitude, power or spatiotemporal localization of the magnetic field supplied by the magnetic coil to control a rate of warming of the ex vivo organ. The magnetic field coil supplies an alternating magnetic field to the ex vivo organ. The method for preserving and recovering biological tissue may include cooling the perfused ex vivo organ to a first temperature prior to scanning the ex vivo organ using magnetic particle imaging. The method for preserving and recovering biological tissue may include cooling the perfused ex vivo organ to a first temperature after scanning the ex vivo organ using magnetic particle imaging. The magnet-responsive colloid may include magnetic particles have a particle size of from about 1 nm to about 10 microns.

A biological tissue preservation composition is disclosed. The biological tissue preservation composition includes a cryoprotectant formulation, and a plurality of magnetic particles dispersed within the cryoprotectant formulation. Implementations of the biological tissue preservation composition may include where the cryoprotectant formulation includes ethylene glycol. The ethylene glycol is present in an amount of from about 10% to about 50% based on a total weight of the biological tissue preservation composition. The plurality of magnetic particles may include iron. The plurality of magnetic particles have a particle size of from about 1 nm to about 10 microns. The plurality of magnetic particles are present in an amount of from about 0.01% to about 10% based on a total weight of the biological tissue preservation composition. The plurality of magnetic particles have favorable magnetic, thermal and colloidal properties including saturation magnetization, coercivity, hysteresis area, high specific loss power, high magnetic particle imaging intensity and resolution, blocking temperature, curie temperature, colloidal stability in the temperature range of −150 °C to 40 °C.

A system for recovering biological tissue is disclosed. The system for recovering biological tissue includes a radiofrequency (100-440 khz) alternating magnetic field coil, dc gradient field coils for spatiotemporal localization of an effective alternating magnetic field region within the biological tissue volume. The system also includes a magnetic particle imaging scanner. The system also includes a multi-input multi-output adaptive feedback control device connected to an input of an alternating magnetic frequency coil.

Implementations of the system for recovering biological tissue may include where the feedback control device further includes a temperature probe. The temperature probe is a fiberoptic temperature probe having one or more gallium arsenide (GaAs) sensors along a length of the temperature probe. The output from the magnetic particle imaging scanner may include imaging data, processed in a separate workstation to generate 3D models of the biological tissue and particle distribution as inputs to finite element modeling-based simulations. The output from the magnetic particle imaging scanner may include a visual display, processed separately in a workstation for post-processing, 3D model generation, computer simulation, or a combination thereof. The radiofrequency alternating magnetic field coil is configured to provide heat to a biological tissue sample. The biological tissue sample is perfused with a biological tissue preservation composition. The biological tissue preservation composition may include, a cryoprotectant formulation, and a magnetic particle dispersed within the cryoprotectant formulation. The cryoprotectant formulation may include organic solvents including ethylene glycol. The ethylene glycol is present in an amount of from about 10% to about 50% based on a total weight of the biological tissue preservation composition. The magnetic particle may include ferrite-based particles. The magnetic particle has a particle size of from about 1 nm to about 10 microns. The magnetic particle is present in an amount of from about 0.01% to about 10% based on a total weight of the biological tissue preservation composition. The magnetic particle has favorable magnetic, thermal and colloidal properties including saturation magnetization, coercivity, hysteresis area, high specific loss power, high magnetic particle imaging intensity and resolution, blocking temperature, curie temperature, colloidal stability in the temperature range of −150 °C to 40 °C.

The features, functions, and advantages that have been discussed can be achieved independently in various implementations or can be combined in yet other implementations further details of which can be seen with reference to the following description.

### BRIEF DESCRIPTION OF THE DRAWINGS

The accompanying drawings, which are incorporated in and constitute a part of this specification, illustrate embodiments of the present teachings and together with the description, serve to explain the principles of the disclosure. In the figures:

FIG. 1 is a schematic illustrating a system for magnetic-based image-guided magnetic warming of a cryopreserved organ using temperature-feedback control, in accordance with the present disclosure.

FIG. 2 is a block diagram illustrating a portion of a process for a feedback-controlled magnetic rewarming system, in accordance with the present disclosure.

FIG. 3 is a flowchart illustrating a method for preserving and recovering biological tissue, in accordance with the present disclosure.

It should be noted that some details of the figures have been simplified and are drawn to facilitate understanding of the present teachings rather than to maintain strict structural accuracy, detail, and scale.

### DETAILED DESCRIPTION

Reference will now be made in detail to exemplary embodiments of the present teachings, examples of which are illustrated in the accompanying drawings. Wherever possible, the same reference numbers will be used throughout the drawings to refer to the same, similar, or like parts.

Issues associated with preserving organs can include those related to the volume of the organs as they scale. For example, ice crystals are a primary issue with this system, measurement, and method as described herein. Temperature gradients across the volume when using typical warming procedures in a “bath” can result in cracking. This includes issues associated with internal heating as localized within the blood vessels. Therefore, an additional feedback control algorithm is needed to modulate the power. A system or device is provided that integrates these functions as well as the method of cryopreservation with the use of magnetic particle-based warming. Imaging and feedback control are integral components of the measurement and rewarming system. The current clinical standard for organ preservation is storage on ice, in which a heart may survive for six hours, a liver may survive for approximately 12 hours, or a kidney may survive for approximately 18 hours. Under these conditions, up to 20% of organs may be discarded due to issues with storage and/or transportation. Examples of the present disclosure provide a movement from an emergency driven system to one unconstrained by time or geography. By adopting cryopreservation, a reduction of metabolic activity can result, slowing down time for degradation and other issues associated with organ preservation and storage. While storing at or below 4 °C, in the range of ice storage, ice crystals can form. By introducing the perfusion of cryoprotecting agents (CPA) can enable balancing toxicity and the parameters of cooling and rewarming. Rapid cooling can lock an organ perfused with CPAs into a glassy state, and therefore be stored for months, as there is no molecular diffusion. An additional challenge is subsequent rewarming of the ex vivo organ, as it adds much higher constraints to avoid ice crystals and cracking. Convective rewarming cannot achieve the required rate to avoid the formation of ice. Magnetic particle heating is based on hysteresis of the plurality of magnetic particles when subjected to an alternating magnetic field. Magnetic field (AC) direction changes and the moments of perfused magnetic nanoparticles can allow a more controlled manipulation of the temperature of the MNPs and therefore the rewarming temperature, as well as the rate of rewarming. By contrast, a constant magnetic field could cause non-uniformity in rewarming.

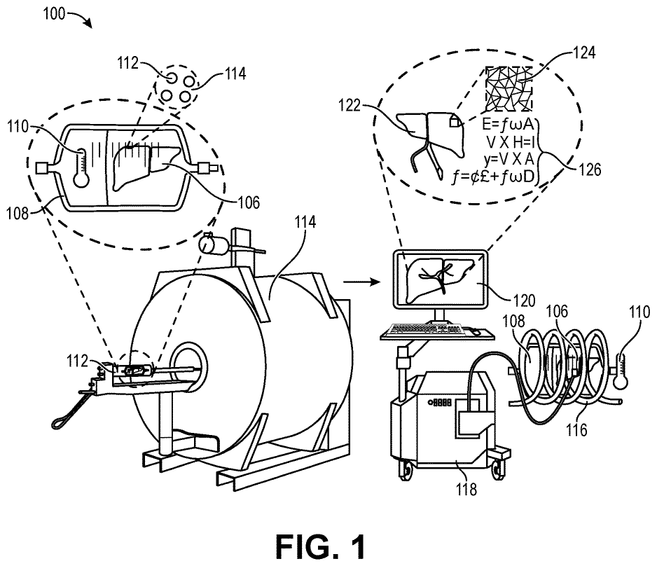

The present disclosure provides for the development of a scalable automated rewarming method for recovering intact cryopreserved organs, in certain examples, livers, for transplantation using rapid and uniform magnetic rewarming based on imaging-guided computational planning and real-time temperature feedback-control. Methods and systems of the present would prevent physical damage of the organ due to ice nucleation, and tissue cracking from thermal gradients during recovery from a cryopreserved state. The motivation for such a scalable organ preservation technology is that storage at lower than hypothermic temperatures (0-4 °C) can increase storage times of transplantable organs beyond current limits, for example, 12 hours for livers on ice, and would therefore, increase the supply of transplantable organs to reduce waiting lists. With current technology, successful organ cryopreservation for transplantation is impeded by the lack of uniform and rapid rewarming methods which enable (viable) organ recovery from a cryopreserved state. Convective warming methods are inadequate and fail because thermal gradients generated between the surface and the interior of the organ cause cracking, and slower warming rates in the interior of the organ produce ice nucleation and growth. With increasing volume and/or shape anisotropy of a cryopreserved organ, such as liver, risks of physical damage during rewarming increase because (i) the probability of ice nucleation increases with volume, and (ii) non-uniform convective heating increases with surface area. These issues can be addressed by (i) using a magnetic rapid rewarming method, which disperses magnetic nanoparticles as nano-heaters within the organ vasculature, and warms the organ internally and externally, by applying an external RF magnetic field, (ii) applying image-guided computational planning and feedback control to the magnetic warming method for real time optimization of the uniformity and speed of rewarming. The warming method is based on hysteresis heating from magnetic nanoparticles dispersed throughout the organ. Here, an image-guided feedback control method is utilized which would allow for clinically scaling of this technology to humans as shown in FIG. 1.

FIG. 1 is a schematic illustrating a system for magnetic-based image-guided magnetic warming of a cryopreserved organ using temperature-feedback control, in accordance with the present disclosure. FIG. 1 illustrates a system for magnetic-based magnetic warming of a cryopreserved organ 100, also referred to as a system for recovering biological tissue 100 including a radiofrequency (100-440 kHz) alternating magnetic field coil 116, which may further include DC gradient field coils for spatiotemporal localization of the effective alternating magnetic field region within a biological tissue 106 volume a magnetic particle imaging (MPI) scanner 114. An MPI scanner or imager can detects a tracer material within the biological tissue 106. The technology detects only the tracer material does not see or interact with the biological tissue 106. The MPI scanner provides a strong magnetic field gradient inside the imaging volume by a main magnet. With the magnetic particle imaging (MPI) scanner 114, an initial set of images of a biological tissue 106 sample is recorded. The biological tissue 106, as shown, is a cryopreserved liver held in a container for organ storage 108, where a temperature monitor 110 is used to monitor the temperature at various places within the use of the system 100 and while practicing the method of preserving and recovering biological tissue 106. The temperature probe 110 can include a fiberoptic temperature probe having one or more GaAs sensors along the probe length. Alternate real-time temperature measurements can include 3D thermometry from the MNPs in MPI imaging which utilizes temperature-dependent magnetic relaxation dynamics of the MNPs. It should be noted that rewarming an ex vivo organ using feedback temperature control is a feature provided by the methods and systems of the present disclosure. In certain examples, it may be considered that an effective alternating magnetic field can be more precisely controlled using a feedback control system such as those described herein to deliver, when subjecting magnetic particles possessing a hysteresis effect heating capability to produce the well-controlled heating necessary for ex vivo organ rewarming. The container for organ storage 108 is placed in a carrier 112 that is introduced into the image scanner 114 to generate image data 122. Prior to imaging, the biological tissue sample 106 is perfused with a biological tissue preservation composition including a cryoprotectant formulation 104 and a plurality of magnetic particles 102 dispersed within the cryoprotectant formulation 104. In illustrative examples, the cryoprotectant formulation 102 includes organic solvents including ethylene glycol, for example. Other examples of organic solvents used in cryoprotectant formulations which can be used herein include propylene glycol, formamide, dimethyl sulfoxide (DMSO), polyethylene glycol (PEG), 2,3 butanediol, N-methylformamide, polyvinylpyrrolidone (PVP), polyvinyl alcohol (PVA), commercially available CPAs including M22 (21st Century Medicine Inc), or combinations thereof. The solvent or ethylene glycol is present in an amount of from about 10% to about 50%, or from about 10% to about 40%, or from about 30% to about 40%, based on a total weight of the biological tissue preservation composition 104. The magnetic particles 102 can include, but are not limited to ferrite-based particles. Illustrative magnetic particles can include different (i) compositions-ferromagnetic Co, Ni, Fe, FeCo nanoparticles, ferrimagnetic-iron oxide, cobalt ferrite, manganese ferrite, (ii) shapes-spherical, nanowires/nanorods, nanoflowers (iii) sizes nano to micro scale limited by capillary diameter including commercially available microspheres with embedded iron oxide nanoparticles, (iv) different coatings-PEG, starch, dextran, and certain FDA approved lipid formulations including magnetic cores, or combinations thereof. In examples, the magnetic particle 102 has a particle size of from about 1 nm to about 10 microns, or from about 10 nm to about 1 micron, or from about 50 nm to about 100 nm. In certain examples, the particle size of the magnetic particle 102 may be limited by vessel dimensions in the biological tissue sample 106 intended for cryopreservation. The magnetic particle 102 is present in an amount of from about 0.01% to about 10% based on a total weight of the biological tissue preservation composition. The magnetic particle 102 has favorable magnetic, thermal and colloidal properties including saturation magnetization, coercivity, hysteresis area, high specific loss power, high magnetic particle imaging intensity and resolution, blocking temperature, curie temperature, and colloidal stability in the temperature range of −150 °C to 40 °C. The cryoprotectant formulation can include ethylene glycol, wherein the ethylene glycol is present in an amount of from about 10% to about 50% based on a total weight of the biological tissue preservation composition. Illustrative examples include where the magnetic particle comprises iron, or having a particle size of from about 1 nm to about 10 microns, or where the magnetic particle is present in an amount of from about 0.01% to about 10% based on a total weight of the biological tissue preservation composition, or a combination thereof. Further properties of the magnetic particle can include where the magnetic particle in the biological tissue preservation composition has favorable magnetic, thermal and colloidal properties including saturation magnetization, coercivity, hysteresis area, high specific loss power, high magnetic particle imaging intensity and resolution, blocking temperature, curie temperature, colloidal stability in the temperature range of −150 °C to 40 °C, or a combination thereof. In general, any magnet-responsive colloid would be applicable to the systems and methods of the present disclosure, including the aforementioned nanoparticles. For example, other magnetic materials, such as cobalt, nickel, samarium, and the like would be applicable in addition to ferrite-based materials. In other examples, Curie temperature control can be an alternate strategy for rewarming applications, which by alloying with different magnetic materials having varied Curie temperatures, can provide further options for Curie temperature control of heating or warming in order to prevent overheating.

Next, the cryopreserved biological tissue 106 is frozen until such time as the biological tissue 106 is necessary for transplantation or for other purposes. When, reheated, a multi-input multi-output adaptive feedback control device 118 connected to an input of the alternating magnetic frequency coil 116 is used as part of the system for recovering biological tissue 100. the radiofrequency alternating magnetic field coil 116 is configured to provide heat to the biological tissue sample 106 during the rewarming process. The heating of the plurality of magnetic particles is based on hysteresis of the plurality of magnetic particles when subjected to an alternating magnetic field and accomplished by a coupling of the effective alternating magnetic field with the magnet-responsive colloid or a plurality of magnetic particles. The alternating magnetic frequency coil 116 is connected electrically and by a suitable data transmission connection to a feedback control unit 118 and image guided feedback module 120, illustrated as a monitor, configured for artificial intelligence (AI)-based 3D segmentation 122, segmented image meshing 124, and finite element predictive modeling 126. The feedback control device 118 also includes a temperature probe 110 for monitoring the rewarming process for the biological tissue 106. The output from the magnetic particle imaging scanner 114 includes initial and in-process imaging data 122, processed in a separate workstation to generate 3D models 126 of the biological tissue 106 and evaluate magnetic particle 102 distribution as inputs to finite element modeling-based simulations 126, in some examples with the use of meshing 124 of the 3D models 126 based on the imaging data 122. The output from the magnetic particle imaging scanner 114 further includes a visual display 120 and provides an image guided feedback model, processed separately in a workstation for post-processing, 3D model generation, computer simulation, or a combination thereof. On one experimental scale, a rodent liver will be perfused, ex vivo, with cryoprotectants (CPAs) and magnetic nanoparticles (MNPs) followed by an application of the application of magnetic particle imaging (MPI) to determine the distribution of the nano-heaters. Image segmentation and finite element simulations of the magnetic excitation and heat transfer analysis in the liver. From the simulations, the temperature feedback-control warming parameters will be predicted as necessary to avoid damage from ice and cracking, which can be assessed experimentally in rodent livers and demonstrate scale up through simulations in human organs. This warming process, as described herein, can be considered automated warming, as the warming uses real-time, in-process feedback from temperature data, along with experimental data and computer simulations, as inputs to guide the system to warm the organs from a first temperature to a second temperature. Thus, the warming can be accomplished using an automated feedback control.

For experimental verification purposes, the determination of a distribution of magnetic nanoparticles (MNP) or magnet-responsive particles as heat sources in a rodent liver, ex vivo, will be conducted using magnetic particle imaging (MPI), following perfusion with a cryoprotectant agent (CPA) with MNPs at hypothermic temperatures, ranging from about 0 °C to about 4 °C. The characterization of MNP distribution within an organ following perfusion is of interest because the uniformity, which can impact thermal gradients, and the rate of the rewarming process depend directly on the distribution of the MNP heat sources within the organ and the power deposited when exposed to radio frequency (RF) alternating magnetic fields (AMFs). Traditional imaging methods, such as micro computed tomography (μCT) and magnetic resonance imaging (MRI) are either too damaging to the organ, due to the exposure of the organ to high energy X-rays or offer poor resolution and poor quantification of an MNP concentration. However, as MPI directly measures magnetization, MNPs provide a tracer yielding high spatial resolution of the MNPs with quantitative MNP concentration within the organ without damage when MPI is used. Known organ hypothermic (0 to 4 °C) perfusion methods can be used to distribute the MNPs and CPAs within the liver. Synomag (Micromod Partikeltechnologie, GmbH) MNPs (multi-crystallite multi-domain), which have high MPI contrast and high specific loss power (SLP), can be used for these processes. PEG-coatings on Synomag MNPs can further be used to ensure colloidal stability in CPAs. Ethylene glycol (EG)-based CPAs can be used for perfusion, as they possess documented thermal properties, physically attainable cooling and warming rates to avoid ice crystallization, and have shown biocompatibility with hepatocytes, spheroids and livers. A Momentum MPI scanner, and compatible hyperthermia platform, HYPER prototype (Ivkov lab), capable of integration with MPI, are unique capabilities that allow truly integrated imaging-guided heat treatments. The MPI-HYPER combination can be used for rewarming cryopreserved tissues, providing a translational and scalable solution to this application. As a part of the procedure, MRI images of the liver can be co-registered with MPI, to determine the MNP distribution relative to anatomical structures, which are used in the analysis in MPI-guided electromagnetic (EM) and heat transfer simulations of rewarming to optimize AMF amplitude and power feedback control protocols.

The use of MPI-guided electromagnetic (EM) and heat transfer simulations of rewarming to optimize AMF amplitude and power feedback control protocols addresses technical challenges to successfully rewarming large organs from a cryopreserved state precludes their use in organ transplantation procedures. The objective of this aim is to obtain, through simulations, optimized rewarming parameters of applied AMF-field amplitude modulation and/or power modulation, for achieving uniform and rapid rewarming of the cryopreserved liver without damage from cracking or ice crystallization. In the simulations, the minimum cooling rate (critical cooling rate, CCR −1 °C/min) and rewarming rate (critical warming rate, CWR 50 °C/min), can be specified to avoid ice crystallization, based on the thermal properties and documented differential scanning calorimetry (DSC) measurements of the CPA used, in certain examples, 40% EG. Upper thresholds can also be placed on thermal gradients, for example a dT/dr≤5 °C/cm, to avoid cracking, based on the thermomechanical properties of 40% EG in glassy state. MPI and MRI co-registered liver images, are imported into Mimics Medical 24.0 (Materialise) for segmentation and meshing. The segmentation of major hepatic blood vessels, such as portal vein, hepatic artery, major blood vessels, and the sinusoids can be accomplished using voxel intensity-based thresholding. Meshing and smoothing of the 3D liver and vasculature are optimized using Materialise 3-Matic software. The smoothed and meshed 3D liver data is exported to COMSOL Multiphysics for coupled EM and heat transfer simulations. For scaling the image contrast from the MNPs to iron concentration (cFe), MPI signal-intensity calibration is performed against ICP-MS Fe standards. An SLP value, based on experimental measurement of SLP of Synomag MNPs in CPA, will be attributed to the MNP heat sources in the vasculature within the model. Additionally, Joule heating from eddy-currents will be accounted for by defining the temperature-dependent electrical conductivity of the CPA-perfused liver tissue.

Convective boundary conditions are assigned to the container holding the vitrified liver. Proportional-integral-derivative (PID)-based feedback control and machine learning methods are employed to maintain warming rates above the critical warming rate (>CWR) and dT/dr below (<) a threshold gradient. The Johnson-Avrami model can be used to then quantitively predict the volume fraction of ice crystallization during rewarming, and generate a 3D strain map to quantify the probability of cracking. Process validation and verification are performed experimentally on containers containing CPA only in a temperature ranging from −150 °C to 0 °C. Finally, application of the rewarming methods resulting from validated simulations to vitrified rodent livers is done. Multipoint thermometry using 4 fiber-optic sensors (FISO Evolution, FISO) are used to measure the rewarming rates. temperature feedback control is then experimentally implemented, using FPGA-based PID and pulse width modulation (PWM) controller (LabVIEW, National Instruments) in agreement with known methods. hepatocyte viability (AO/PI, histology, FACS), liver function (bile production, O2 consumption) and injury (ALT, LDH, CD31) are assessed. Convectively warmed livers are used as negative controls. Statistical analysis for correlating predicted damage from ice and cracking vs functional endpoints are also performed.

The process and system described herein provides an organ rewarming process flow that minimizes or eliminates physical damage to rodent livers from ice crystallization and cracking during rewarming from the cryopreserved vitrified state, with scale-up computational model(s). Validation of these findings can be gained with successful transplantation in live rodents, demonstrating a first and meaningful translational step forward for cryopreservation of large tissues and organs. An additional intermediate outcome would be extension of this technology to liver sub-systems such as hepatocytes, tissue slices and liver lobes for pharmacological research. A longer-term outcome would be success in transplantation of cryopreserved livers and other organs in humans. This would, consequently, allow the practical implementation of organ banking in the cryopreserved state with substantially extended storage times over traditional cold storage. Success in this effort can lead to global changes towards fair organ allocation.

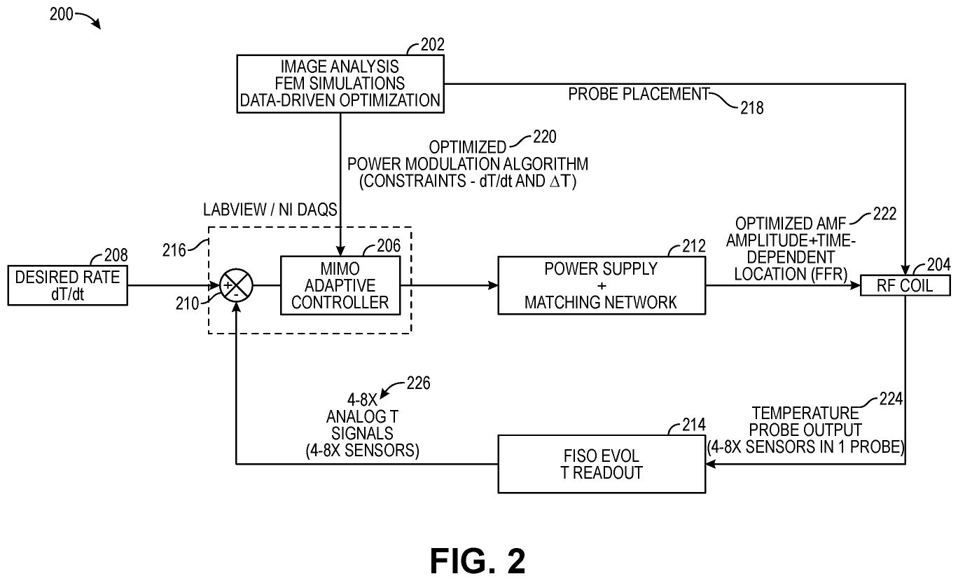

FIG. 2 is a block diagram illustrating a portion of a process for a feedback-controlled magnetic rewarming system, in accordance with the present disclosure. A process for a feedback-controlled magnetic rewarming system 200 includes image analysis, finite element modeling (FEM) simulations and data-driven optimization 202, which provides output information to operation of the radio frequency (RF) coil 204, and to a multi-input, multi-output (MIMO) adaptive controller 206. A desired rate of warming (dT/dt) 208 is provided via calculation 210, which is the calculated arithmetic difference or error between desired heating rate and current measured heating rate. The difference and other similar calculations are generally evaluated digitally using a computer that is connected to a field-programmable gate array (FPGA) controller. The desired rate of warming (dT/dt) 208 is provided to the multi-input, multi-output (MIMO) adaptive controller 206 as well. The adaptive controller 206 provides its respective inputs to a power supply and matching network 212, which operates the radio frequency (RF) coil 204 to achieve an optimized alternating magnetic field (AMF) amplitude and time-dependent location field free region (FFR) 222. Additional feedback to the operation of the radio frequency (RF) coil 204 is provided by a temperature probe placement 218 that monitors the temperature of an organ or biological tissue samples during rewarming to ensure the desired rate of warming (dT/dt) 208 is met. This desired rate of warming (dT/dt) 208 is also combined with the image analysis, finite element modeling (FEM) simulations and data-driven optimization 202 to determine an optimized power modulation algorithm 220, which is constrained by dT/dt and spatial difference in temperature (AT), which is also provided to the power supply and matching network 212 via the adaptive controller 206. The operation of the adaptive controller 206, interface 210 occur within a LabVIEW/National Instruments (NI) data acquisition system (Daqs) 216. The operation of the radio frequency (RF) coil 204 further provides a temperature probe output 224 which records and transmits temperature measurement data to a FISO™ Evolution temperature (T) readout 214, which can include from multiple (4-8x) sensors in a single temperature probe 224, and these 4-8 analog temperature signals 226 are passed into the interface 210 from the 4 to 8 sensors in the temperature probe output 224 to complete the feedback loop and monitoring of the temperature while a biological tissue sample or organ undergoes the rewarming process of the present disclosure. This temperature feedback control system as described herein provides an effective alternating magnetic field to the MNP/CPA composition within an organ to produce the well-controlled heating necessary for ex vivo organ rewarming.

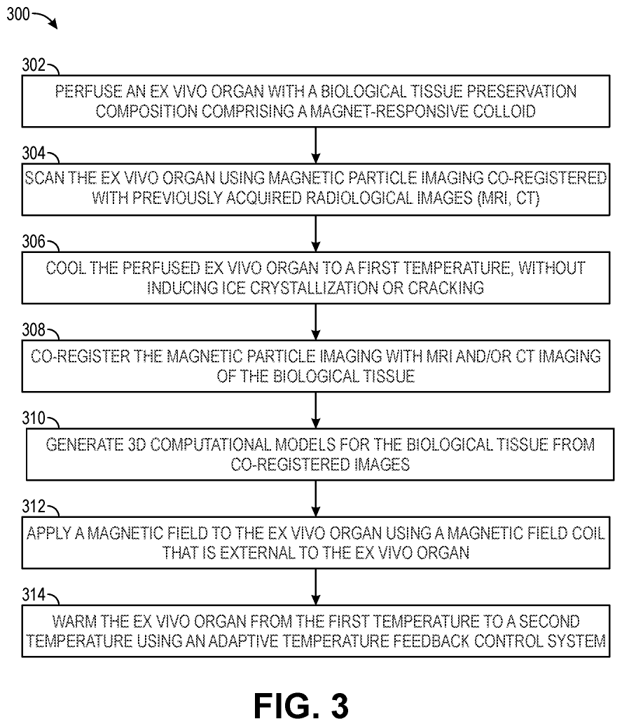

FIG. 3 is a flowchart illustrating a method for preserving and recovering biological tissue, in accordance with the present disclosure. A method for preserving and recovering biological tissue 300, is disclosed including a step to perfuse an ex vivo organ with a biological tissue preservation cryoprotectant mixture comprising magnet-responsive particles or magnetic particles 302, followed by a scanning of the ex vivo organ using magnetic particle imaging co-registered with previously acquired radiological images (MRI, CT) 304. Next, the perfused ex vivo organ is cooled to a first temperature 306, without inducing ice crystallization or cracking. This first temperature can range from about 0 °C to about −150 °C. The scanning of the ex vivo organ using magnetic particle imaging 304 to generate one or more images representing a distribution of magnet-responsive colloid within the ex vivo organ may be repeated after cooling, in certain examples of the present disclosure. Again, the method for preserving and recovering biological tissue 300 may include an additional step of co-registering the magnetic particle imaging with MRI and/or CT imaging of the biological tissue 308, followed by generating 3D computational models for the biological tissue from co-registered images 310, applying a magnetic field to the ex vivo organ using a magnetic field coil that is external to the ex vivo organ 312, and warming the ex vivo organ from the first temperature to a second temperature 314. This second temperature target for the rewarming process can range from about 0 °C to about 40 °C.

The method for preserving and recovering biological tissue 300 can include where the biological tissue preservation cryoprotectant composition comprises organic solvents including ethylene glycol, sugars, antifreeze proteins, and a magnet-responsive colloid, or a combination thereof. The magnet-responsive colloid can include, but is not limited to, magnetic particles having a particle size of from about 1 nm to about 10 microns. Additional steps of the method for preserving and recovering biological tissue 300 can include storing the ex vivo organ at or below the first temperature, maintaining the ex vivo organ at or below the first temperature, and transporting the ex vivo organ from a first geographical location to a second geographical location. This allows for extended time allowance between an organ harvesting and preservation operation and eventual use or transplantation. In certain examples, portions or steps of the method for preserving and recovering biological tissue 300 can occur in a first geographical location and be performed by a first entity, while other portions or steps of the method for preserving and recovering biological tissue 300 can occur in a first geographical location by a first entity or alternatively in a second geographical location by a second entity. It is envisioned, for example, that process steps associated with removal and cryopreservation of an organ or biological tissue sample can be accomplished in one location by a first entity or organization, while the rewarming and/or transplantation or use of the organ or biological tissue sample can be conducted at a second location by a second entity or organization.

The method for preserving and recovering biological tissue 300 may include performing one or more finite element modeling-based simulations of coupled electromagnetic and heat transfer in 3D models of the biological tissue using the one or more generated images, and subsequently inputting the optimization results of the one or more simulations including the proportional, integral and derivative gains into a multi-input multi-output (MIMO) adaptive temperature feedback control system to control one or more parameters of the magnetic field. The one or more parameters of the magnetic field may include frequency, amplitude, power, spatial and temporal localization of the effective alternating magnetic field relative to one or more biological tissue contours, or a combination thereof. Certain examples of the method for preserving and recovering biological tissue 300 can include steps to monitor temperature of the ex vivo organ while applying the magnetic field and modulating an amplitude, power or spatiotemporal localization of the alternating magnetic field supplied by the magnetic coil to control a rate of warming of the ex vivo organ. The magnetic field coil can supply an alternating magnetic field to the ex vivo organ. Without being exhaustive, there are multiple known methods of create spatially confined fields to provide spatiotemporal control of the AMF, such as the use of opposing static magnets to create a FFR, among others.

In illustrative examples of the method for preserving and recovering biological tissue 300, the method may include perfusing the ex vivo organ with a biological tissue preservation composition including a magnet-responsive colloid prior to scanning using magnetic particle imaging. The perfused ex vivo organ may be cooled or warmed at different rates. For example, the perfused ex vivo organ can be cooled to the first temperature at a cooling rate from about 0.5 °C/min to about 20 °C/min or at a rate of warming is greater than 50 °C/min. Certain examples of the method for preserving and recovering biological tissue 300 include scanning an ex vivo organ using magnetic particle imaging to generate one or more images representing a distribution of a magnet-responsive colloid within the ex vivo organ, applying a magnetic field to the ex vivo organ using a magnetic field coil that is external to the ex vivo organ, and warming the ex vivo organ from a first temperature to a second temperature. Alternatively, the steps of performing one or more finite element modeling-based simulations of coupled electromagnetic and heat transfer in 3D models of the biological tissue using the one or more generated images, inputting the optimization results of the one or more simulations including the proportional, integral and derivative gains into a multi-input multi-output (MIMO) adaptive temperature feedback control system to control one or more parameters of the magnetic field may be included where one or more parameters of the magnetic field comprise frequency, amplitude, power, spatial and temporal localization of the alternating magnetic field relative to the biological tissue boundaries or a combination thereof. Temperature monitoring of the ex vivo organ may be done while applying the magnetic field, and modulating an amplitude, power or spatiotemporal localization of the magnetic field supplied by the magnetic coil to control a rate of warming of the ex vivo organ may be conducted during the method 300, while the magnetic field coil supplies an alternating magnetic field to the ex vivo organ.

In an illustrative example of the method for preserving and recovering biological tissue, all of the steps of perfusing an ex vivo organ with a biological tissue preservation composition comprising a magnet-responsive colloid, scanning the ex vivo organ using magnetic particle imaging; cooling the perfused ex vivo organ to a first temperature. scanning an ex vivo organ using magnetic particle imaging to generate one or more images representing a distribution of magnet-responsive colloid within the ex vivo organ, co-registering the magnetic particle imaging with MRI and/or CT imaging of the biological tissue generating 3D computational models for the biological tissue from co-registered images, applying a magnetic field to the ex vivo organ using a magnetic field coil that is external to the ex vivo organ, and warming the ex vivo organ from the first temperature to a second temperature occur in a single location and are conducted by a single entity or organization.

The method may further include performing one or more finite element modeling-based simulations of coupled electromagnetic and heat transfer in 3D models of the biological tissue using the one or more generated images, inputting the optimization results of the one or more simulations including the proportional, integral and derivative gains into a multi-input multi-output (MIMO) adaptive temperature feedback control system to control one or more parameters of the magnetic field, monitoring temperature of the ex vivo organ while applying the magnetic field; and modulating an amplitude, power or spatiotemporal localization of the magnetic field supplied by the magnetic coil to control a rate of warming of the ex vivo organ, cooling the perfused ex vivo organ to a first temperature prior to scanning the ex vivo organ using magnetic particle imaging, cooling the perfused ex vivo organ to a first temperature after scanning the ex vivo organ using magnetic particle imaging, or any combination of steps as described herein in modified order of steps.

While the present teachings have been illustrated with respect to one or more implementations, alterations and/or modifications may be made to the illustrated examples without departing from the spirit and scope of the appended claims. For example, it may be appreciated that while the process is described as a series of acts or events, the present teachings are not limited by the ordering of such acts or events. Some acts may occur in different orders and/or concurrently with other acts or events apart from those described herein. Also, not all process stages may be required to implement a methodology in accordance with one or more aspects or embodiments of the present teachings. It may be appreciated that structural objects and/or processing stages may be added, or existing structural objects and/or processing stages may be removed or modified. Further, one or more of the acts depicted herein may be carried out in one or more separate acts and/or phases. Furthermore, to the extent that the terms “including,” “includes,” “having,” “has,” “with,” or variants thereof are used in either the detailed description and the claims, such terms are intended to be inclusive in a manner similar to the term “comprising.” The term “at least one of” is used to mean one or more of the listed items may be selected. Further, in the discussion and claims herein, the term “on” used with respect to two materials, one “on” the other, means at least some contact between the materials, while “over” means the materials are in proximity, but possibly with one or more additional intervening materials such that contact is possible but not required. Neither “on” nor “over” implies any directionality as used herein. The term “conformal” describes a coating material in which angles of the underlying material are preserved by the conformal material. The term “about” indicates that the value listed may be somewhat altered, as long as the alteration does not result in nonconformance of the process or structure to the illustrated embodiment. The terms “couple,” “coupled,” “connect,” “connection,” “connected,” “in connection with,” and “connecting” refer to “in direct connection with” or “in connection with via one or more intermediate elements or members.” Finally, the terms “exemplary” or “illustrative” indicate the description is used as an example, rather than implying that it is an ideal. Other embodiments of the present teachings may be apparent to those skilled in the art from consideration of the specification and practice of the disclosure herein. It is intended that the specification and examples be considered as exemplary only, with a true scope and spirit of the present teachings being indicated by the following claims.

What is claimed is:

1. A method for preserving and recovering biological tissue, comprising:
perfusing an ex vivo organ with a biological tissue preservation cryoprotectant composition comprising magnet-responsive particles;
scanning the ex vivo organ using magnetic particle imaging co-registered with previously acquired radiological images (MRI, CT); and
rapidly cooling the perfused ex vivo organ to a first temperature without inducing ice crystallization or cracking;
scanning an ex vivo organ using magnetic particle imaging to generate one or more images representing a distribution of magnetic particles within the ex vivo organ;
co-registering the magnetic particle imaging with MRI and/or CT imaging of the biological tissue generating 3D computational models for the biological tissue from co-registered images;
applying an effective alternating magnetic field to the ex vivo organ using a magnetic field coil that is external to the ex vivo organ; and
warming the ex vivo organ from a first temperature to a second temperature using automated feedback control.

2. The method for preserving and recovering biological tissue of claim 1, wherein the biological tissue preservation cryoprotectant composition comprises organic solvents including ethylene glycol, sugars, antifreeze proteins and a magnet-responsive colloid, or a combination thereof.

3. The method for preserving and recovering biological tissue of claim 1, wherein the magnet-responsive colloid comprises magnetic particles having a particle size of from about 1 nm to about 10 microns.

4. The method for preserving and recovering biological tissue of claim 1, further comprising storing the ex vivo organ at or below a first temperature.

5. The method for preserving and recovering biological tissue of claim 1, further comprising:
maintaining the ex vivo organ at or below the first temperature; and
transporting the ex vivo organ from a first geographical location to a second geographical location.

6. The method for preserving and recovering biological tissue of claim 1, further comprising:
performing one or more finite element modeling-based simulations of coupled electromagnetic and heat transfer in 3D models of the biological tissue using the one or more generated images; and
inputting optimization results of the one or more simulations including proportional, integral and derivative gains into a multi-input multi-output (MIMO) adaptive temperature feedback control system to control one or more parameters of the magnetic field.

7. The method for preserving and recovering biological tissue of claim 6, wherein the one or more parameters of the magnetic field comprise frequency, amplitude, power, spatial and temporal localization of the effective alternating magnetic field relative to biological tissue contours or a combination thereof.

8. The method for preserving and recovering biological tissue of claim 6, further comprising:
monitoring temperature of the ex vivo organ while applying the magnetic field; and
modulating an amplitude, power or spatiotemporal localization of the effective alternating magnetic field supplied by the magnetic coil using an adaptive controller to control a rate of warming of the ex vivo organ.

9. The method for preserving and recovering biological tissue of claim 1, wherein the magnetic field coil supplies an alternating magnetic field to the ex vivo organ.

10. The method for preserving and recovering biological tissue of claim 1, further comprising perfusing the ex vivo organ with a biological tissue preservation composition comprising a magnet-responsive colloid prior to scanning using magnetic particle imaging.

11. The method for preserving and recovering biological tissue of claim 1, wherein the perfused ex vivo organ is cooled to the first temperature at a cooling rate from about 0.5 °C/min to about 20 °C/min.

12. The method for preserving and recovering biological tissue of claim 8, wherein the rate of warming is greater than 50 °C/min.

13. The method for preserving and recovering biological tissue of claim 1, wherein the first temperature is from about 0 °C to about −150 °C and the second temperature is from about 0 °C to about 40 °C.

14. A biological tissue preservation composition, comprising:
a cryoprotectant formulation; and
a plurality of magnetic particles dispersed within the cryoprotectant formulation.

15. The biological tissue preservation composition of claim 14, wherein the cryoprotectant formulation comprises ethylene glycol, present in an amount of from about 10% to about 50% based on a total weight of the biological tissue preservation composition.

16. The biological tissue preservation composition of claim 14, wherein:
the plurality of magnetic particles comprise iron;
the plurality of magnetic particles have a particle size of from about 1 nm to about 10 microns; and
the plurality of magnetic particles are present in an amount of from about 0.01% to about 10% based on a total weight of the biological tissue preservation composition.

17. The biological tissue preservation composition of claim 14, wherein the plurality of magnetic particles have favorable magnetic, thermal and colloidal properties including saturation magnetization, coercivity, hysteresis area, high specific loss power, high magnetic particle imaging intensity and resolution, blocking temperature, curie temperature, colloidal stability in the temperature range of −150 °C to 40 °C.

18. A system for recovering biological tissue, comprising:
A radiofrequency (100-440 kHz) alternating magnetic field coil;
DC gradient field coils for spatiotemporal localization of an effective alternating magnetic field region within the biological tissue volume a magnetic particle imaging scanner; and
a multi-input multi-output adaptive feedback control device connected to an input of an alternating magnetic frequency coil; and wherein:
the biological tissue sample is perfused with a biological tissue preservation composition comprising:
a cryoprotectant formulation; and
a magnetic particle dispersed within the cryoprotectant formulation.

19. The system for recovering biological tissue of claim 18, wherein the feedback control device further comprises a fiberoptic temperature probe having one or more GaAs sensors along a length of the temperature probe.

20. The system for recovering biological tissue of claim 18, wherein:
the output from the magnetic particle imaging scanner comprises imaging data, processed in a separate workstation to generate 3D models of the biological tissue and particle distribution as inputs to finite element modeling-based simulations;
the output from the magnetic particle imaging scanner comprises a visual display, processed separately in a workstation for post-processing, 3D model generation, computer simulation, or a combination thereof; and
the radiofrequency alternating magnetic field coil is configured to provide heat to a biological tissue sample.
